(19) United States
(12) Patent Application Publication
(10) Pub. No.: US 2016/0000326 A1
(43) Pub. Date: Jan. 7, 2016

(54) METHOD OF IMAGING BONE

(71) Applicant: ISIS INNOVATION LTD., Oxford Oxfordshire (GB)

(72) Inventors: Karen ELEY, Oxford (GB); Stephen GOLDING, Oxford (GB)

(21) Appl. No.: 14/769,791

(22) PCT Filed: Feb. 21, 2014
(86) PCT No.: PCT/IB2014/059167

§ 371 Date: Aug. 21, 2015

Related U.S. Application Data
(60) Provisional application No. 61/767,485, filed on Feb. 21, 2013.

(51) Int. Cl. A61B 5/00; G01R 33/48; G01R 33/56; A61B 5/055

(52) U.S. Cl. CPC A61B 5/004; A61B 5/055; G01R 33/4816; G01R 33/5608

(57) ABSTRACT

A method of obtaining an image of bone using magnetic resonance imaging, the method comprising the step of selecting the scanning parameters for the magnetic resonance imaging device, wherein the scanning parameters comprise a low flip angle, a short time to repetition and a short time to echo.

### TECHNICAL FIELD

The present invention relates to a method of imaging bone. The method uses a Magnetic Resonance Imaging device, and is suitable for, but not limited to, the diagnosis of a range of pathologies affecting the head and neck, and three-dimensional (3D) reconstruction of the craniofacial skeleton.

### BACKGROUND

The potential risks associated with ionising radiation are well-known. However, the use of x-ray based imaging, particularly Computed Tomography (CT), continues to rise. During the year 2002-3 the NHS in England reported 1.7 million CT examinations, representing 8% of all x-ray examinations, and 6% of all imaging investigations. By 2009-10 this had risen to over 3.7 million, 13% and 10% respectively.

Some of the largest increases in CT use have been in paediatric diagnosis and adult screening. In children, particularly where imaging is required for benign conditions, the potential deleterious effects of ionising radiation are of greatest concern; organs are more radio-sensitive, and with a greater life expectancy, there is more time in which to develop malignancy. The United Nations Scientific Committee (UNSCEAR) estimated that CT constitutes 5% of all x-ray examinations worldwide while accounting for about 34% of the resultant collective dose. The consensus amongst radiology professionals is that steps should be taken to reverse, or at least arrest radiation exposure from CT.

In an attempt to reduce radiation dose, particularly when imaging the craniofacial region, cone beam CT is increasingly utilised. However, with scanners being installed and used in a rapidly increasing number of dental practices within the UK, such imaging appears to be replacing plain film imaging rather than conventional CT.

Magnetic Resonance Imaging (MRI) offers a non-ionising alternative to CT. However, shortly after its introduction into clinical practice, the poor detail of bone and calcified tissues seen on MRI was reported as a significant limitation of the technique. The superior imaging quality of cortical bone on CT and the ability to create three-dimensional (3D) rendered images of the craniofacial skeleton has maintained CT as the “gold standard” for this region.

The present invention provides an alternative to CT in imaging a range of pathologies, and lends itself to 3D reconstruction of bony structures, thereby eliminating ionising radiation exposure. This is especially important, but not limited to, the craniofacial skeleton where radiation protection is crucial in view of the radiosensitive lens and thyroid gland.

### SUMMARY

According to the present invention there is provided a method of obtaining an image of bone using magnetic resonance imaging, the method comprising the step of selecting the scanning parameters for the magnetic resonance imaging device, wherein the scanning parameters comprise a low flip angle, a short time to repetition and a short time to echo.

The flip angle may be in the range 1° to 10°.

The flip angle may be one of about 1°, 3°, 5° and 7°

The time to repetition may be ≦20 ms.

The time to echo may be ≦20 ms.

The time to echo may be one of the following: 4.6; 9.2; 13.8; and 18.4.

The magnetic resonance imaging step may use a gradient echo sequence.

The time to echo may be “in phase”.

The method of any one of the preceding claims may further comprise performing 3D reconstruction using the image data.

### BRIEF DESCRIPTION OF THE DRAWINGS

Many aspects of the disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 is a diagrammatic illustration of a method embodying the present invention

FIG. 2a is an example of sagittal and axial imaging from an adult in accordance with the present invention.

FIG. 2b is a further example of axial imaging from an adult.

FIG. 2c is a diagram comparing MRI and CT images from a child with sagittal synostosis.

FIG. 3a illustrates biometric measurements on computed tomography for infraorbital foramina distance.

FIG. 3b illustrates biometric measurements on computed tomography for maxilla without teeth.

FIG. 4a illustrates biometric measurements on MRI images using the present invention for orbit height.

FIG. 4b illustrates biometric measurements on MRI images using the present invention mandible height.

FIG. 5a shows an axial MRI image where the flip angle is 1°.

FIG. 5b shows an axial MRI image where the flip angle is 3° and the remaining scan parameters are the same as for the image of FIG. 5a.

FIG. 5c shows an axial MRI image where the flip angle is 7° and the remaining scan parameters are the same as for the image of FIG. 5a.

FIG. 6 depicts axial MRI images of Patient 1, demonstrating normal cortical appearances of the zygomatic bones.

FIG. 7 depicts coronal MRI images of Patient 2, demonstrating inferior displacement of the left orbital floor, and fluid within the left maxillary sinus.

FIG. 8 depicts axial MRI images of Patient 3, demonstrating thinning and loss of the cortex on the medial aspect of the lower margin of the left mandibular keratocyst.

FIG. 9 depicts axial MRI images of Patient 4, demonstrating granuloma within the right maxillary sinus.

FIG. 10 illustrates axial MRI images of Patient 5, demonstrating no evidence of bony expansion of the right superior alveolus;

FIG. 11 illustrates axial MRI images of Patient 6, demonstrating normal cortical morphology, with evidence of trabecular sclerosis at site of previous left retromolar keratocyst.

FIG. 12 illustrates axial MRI images of Patient 7, demonstrating a lesion within the left maxillary alveolus which breaches the lateral cortex with extension into the subcutaneous tissues of the cheek.

FIG. 13 illustrates axial MRI images of Patient 8, with normal appearances of the orbital bones.

FIG. 14a shows axial and coronal MRI imaging from a child with normal cranial sutures.

FIG. 14b shows axial and coronal CT and MRI imaging and 3D reconstructions from a child with metopic synostosis.

FIG. 14c shows axial and coronal CT and MRI imaging and 3D reconstructions from a child with right unicoronal synostosis.

FIG. 14d shows axial and coronal CT and MRI imaging and 3D reconstructions from a child with sagittal synostosis.

### DETAILED DESCRIPTION OF THE DRAWINGS

It should be emphasized that the above-described embodiments are merely examples of possible implementations. Many variations and modifications may be made to the above-described embodiments without departing from the principles of the present disclosure. All such modifications and variations are intended to be included herein within the scope of this disclosure and protected by the following claims.

In the present invention, an MRI sequence can be achieved by utilising a gradient echo sequence with adjustment of the scanning parameters to obtain uniform contrast of the soft tissues, thus enhancing the soft-tissue bone boundary. This enables the clear distinction of bone from the soft tissues and permits 3D rendering of bone. As can be seen in the figures, bone appears as a solid black colour in the resulting images, and the method and images are referred to as “Black Bone” in the description below.

### Method

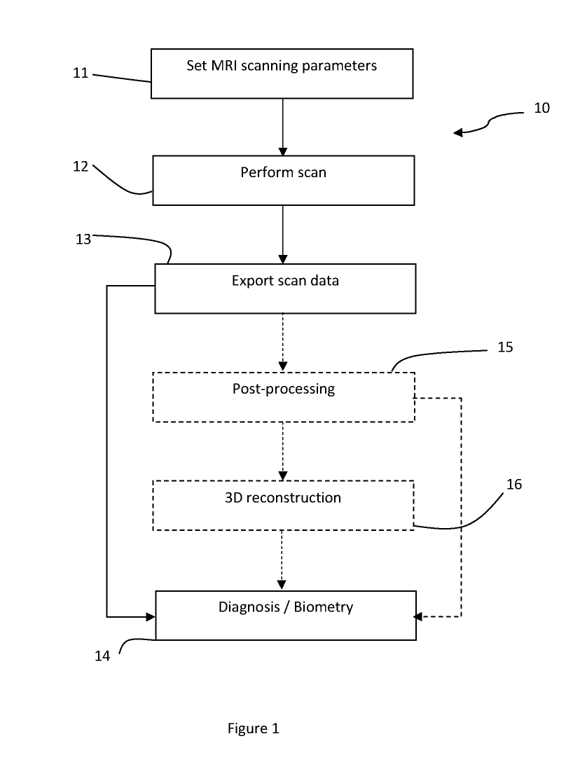

A method of obtaining “Black Bone” images is illustrated at 10 in FIG. 1. At step 11, the MRI scanning parameters are set. In the present invention, it has been found that by using a gradient echo sequence and selecting scanning parameters comprising a low flip angle and short time to repetition (“TR”) and time to echo (“TE”), the contrast between bone and soft tissue can be clearly defined. Bone returns little or no signal in MRI imaging, while the low flip angle minimises the soft tissue return and the short time to repetition and time to echo suppress contrast within the soft tissue. The resulting images show everything outside the bone as uniformly grey as possible, thus enhancing the bone-soft-tissue boundary

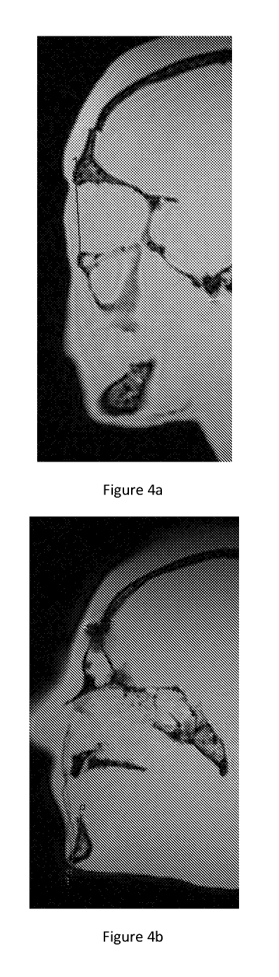

In the present example, a flip angle in the range 1° to 10° provides a reasonable result, most preferably 5°, but as shown in FIGS. 5a to 5c below, acceptable images are achieved at 1°, 3° and 7°.

The time to repetition and time to echo are preferably less than or equal to about 20 ms. In the present case, the time to echo is preferably an even multiple of 2.3 ms and so may be one of the following: 4.6; 9.2; 13.8; and 18.4 ms.

An example of the scanning parameters for a given scan are shown in table 1, where FOV is the field of view and ZIP is the zero fill interpolation.

TABLE 1
“Black Bone” MRI scanning parameters

| Parameter | Value | |
|---|---|---|
| Time repetition | 8.6 | ms |
| Time echo | 4.2 | ms |
| Flip angle | 5 | |
| Slice thickness | 2.4 | mm |
| Slice Spacing | −1.2 | mm |
| Scan FOV | 24 | cm |

| | |
|---|---|
| Phase encode | 256 |
| Frequency encode | 256 |
| Receive bandwidth | 31.25 |
| ZIP | 2, 512 |

Of these parameters the TE, TR and Flip Angle are the key entities to produce the “Black Bone” sequence. The selected TE is considered to be “in phase” and many MRI scanners may utilise this input rather than a discrete value.

At step 12, the scan is performed and imaging captured, and at step 13 the resulting data exported. The data may be exported in any appropriate format, such as DICOM.

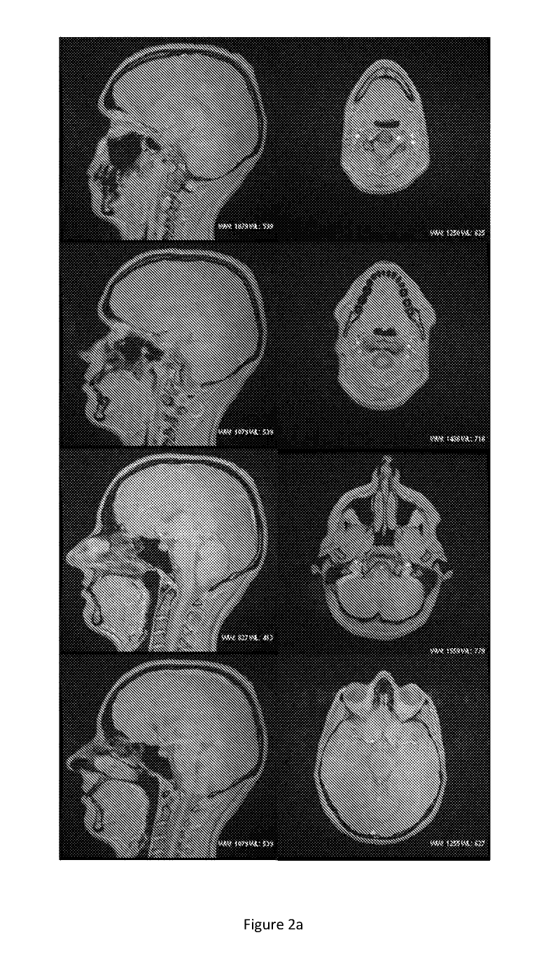

Example images obtained in this way are shown in FIGS. 2a to 2c. FIG. 2a is an example of sagittal and axial imaging from an adult. The method utilised a 1.5 or 3 Telsa system (GE Medical Systems, Milwaukee, Ill., USA), but other such instruments may equally be used. Imaging was performed using the neurovascular array head coil (other coils may equally be used) with the volunteer positioned according to convention for imaging the head. 3D volume acquisition was used although slice acquisition may also be used. Imaging time for an adult skull in the present example is about 4 minutes. FIG. 2b is a further example of axial imaging from an adult. FIG. 2c is a diagram comparing CT (row A) and MRI (row B) images from a child with sagittal synostosis. In each case, the corticol bone appears solid black and the soft tissue is an almost uniform grey.

The images may be used directly for diagnosis or biometric measurements, as shown at step 14 and as demonstrated in some examples below.

Alternatively, as shown in dashed outline, the images may be subject to post-processing as shown at 15 to improve the contrast between bone and soft tissue, and the resulting images may be used directly in diagnosis or provided to a 3D reconstruction system to generate a 3D reconstruction of the imaged skeleton, as shown at step 16. The 3D reconstruction can then be utilised to produce anatomical models using rapid prototyping. Both 3D images and anatomical models are of considerable benefit in diagnosis and surgical planning.

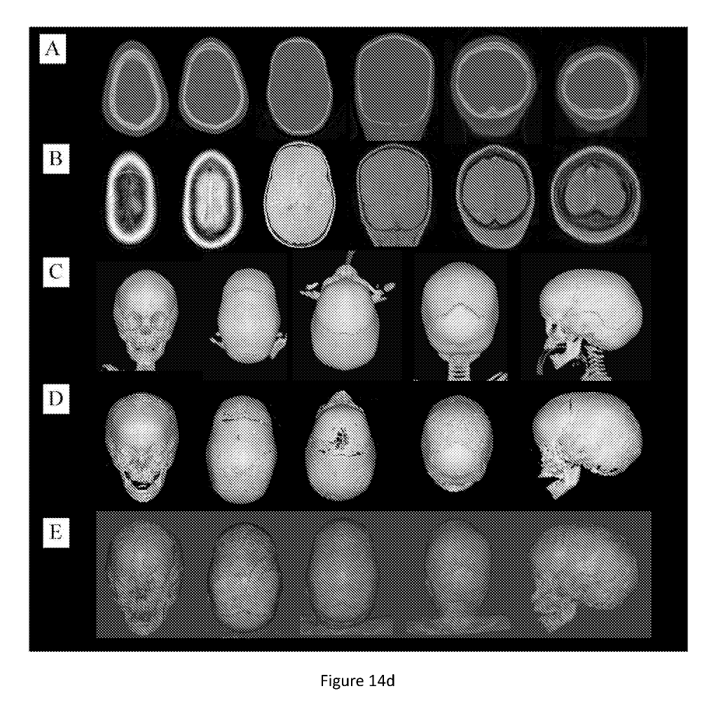

Example of 3D reconstructions are shown below in FIGS. 14b to 14d, produced using the software packages Mimics and Fovia, which use thresholding and volume rendering respectively.

In thresholding, a threshold mask is selected so that the bone is contained within the upper and lower threshold limits. Since this threshold limit will also include the surrounding air, the resulting mask must be edited using a combination of multiple-slice edit and 3D edit functions which result in this technique being time intensive.

In volume rendering, a transfer function is initially applied, thereby assigning a colour and opacity to each pixel, and the soft tissues may be removed from the underlying bone utilising a surface layer removal tool. In the example of visualisation of cranial sutures discussed below, this could be achieved making the soft tissues transparent thus not needing segmentation and images could be created using a transfer function preset very rapidly. Volume rendering techniques with manipulation of the transfer function in addition to segmentation yield the most optimal results.

As will be understood, the present invention has many possible applications, including but not limited to craniosynostosis, salivary gland stones, identification of bony erosion due to malignancy, benign mandibular cysts, facial fractures, cephalometric analysis, and identification of fractures, including facial and cervical fractures.

### Validation

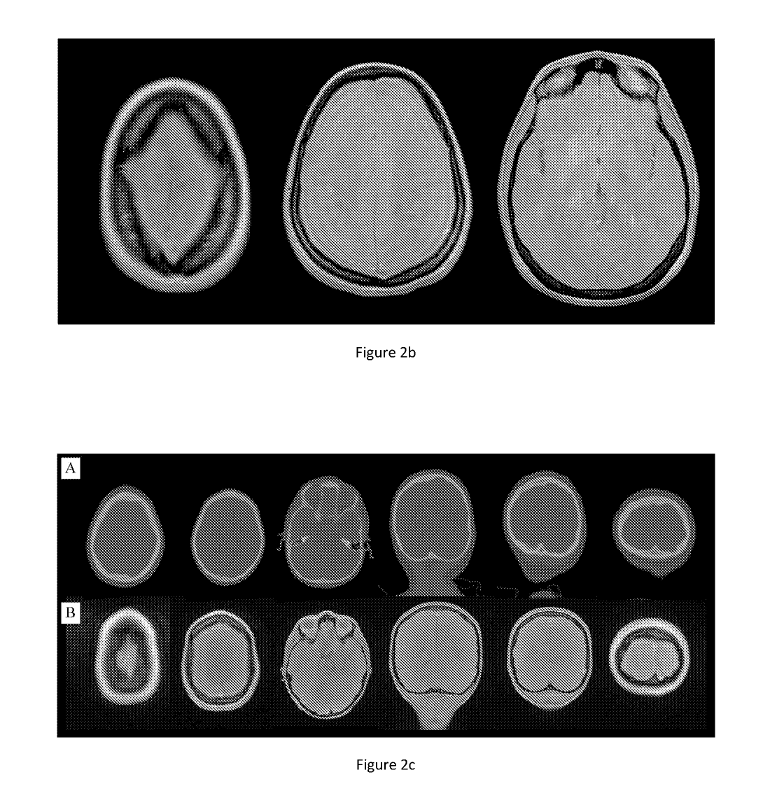

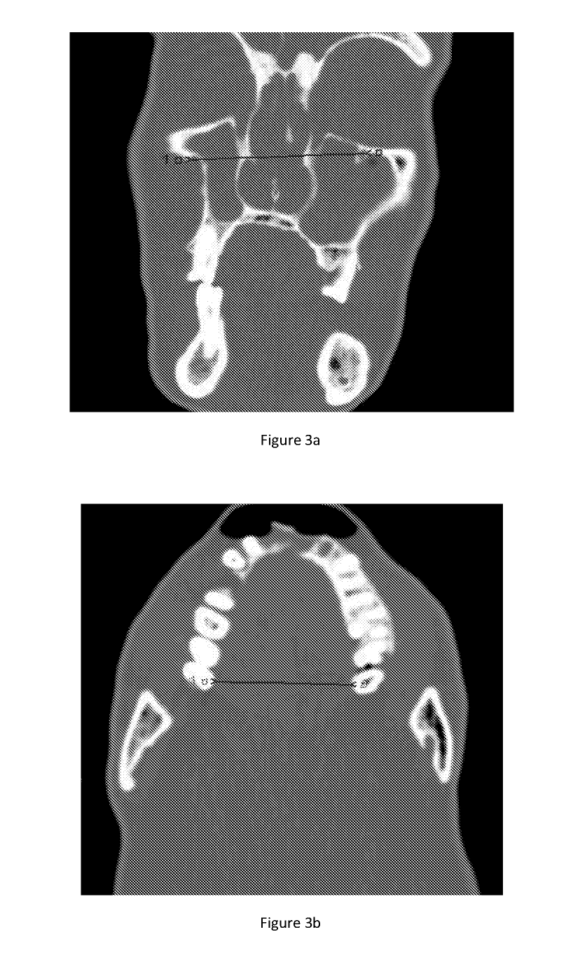

To confirm that the “Black Bone” sequence yielded accurate dimensional data, the biometric accuracy was investigated. This was conducted using a custom made phantom, consisting of a disarticulated skull confined within a water-tight perspex anthromorphic shell filled with copper sulphate solution. Direct anatomical measurements were compared to those obtained from “Black Bone” MRI and CT. Straight line measurements between paired anatomical points which could be identified directly on the phantom, and on both Black Bone MRI and CT (Table 2, FIGS. 3a to 4b) were used. These included the distance between paired foramina, and maximum heights and widths of bony structures.

TABLE 2
Anatomical measurements completed
to determine biometric accuracy

| | Imaging |
|---|---|
| Anatomical Measurement | Plane |
| Maximum cranial-caudal aperture of the right orbit (FIG. 3) | Sagittal |
| Maximum cranial-caudal aperture of the left orbit | Sagittal |
| Maximum height of the mandible from chin point in the midline | Sagittal |
| (FIG. 3) | |
| Maximum coronal dimension of the skull at the disarticulation | Coronal |
| point (cut surface) | |
| Maximum coronal dimension of the skull cap at the | Coronal |
| disarticulation point (cut surface) | |
| Distance between the lateral most aspect of the paired | Coronal |
| infraorbital foramen (FIG. 2) | |
| Maximum cranial-caudal aperture of the piriform aperture | Coronal |
| Maximum coronal aperture of the piriform aperture | Coronal |
| Distance between the lateral most aspect of the paired mental | Coronal |
| foramen (on both axial and coronal views) | |
| Distance between the lateral most aspect of the paired | Axial |
| mandibular condyles | |
| Maximum coronal dimension of the posterior hard palate | Axial |
| between the most posterior identifiable molar teeth (FIG. 2) | |
| Maximum coronal distance between the lateral surfaces of the | Axial |
| paired posterior upper molar teeth | |

For the direct measurements on the phantom vernier callipers were used (resolution 0.01 mm; accuracy: <100 mm±0.02 mm; 100-200 mm±0.03 mm) with each measurement repeated and recorded twenty times on two separate occasions with a time interval of two weeks by one assessor.

“Black Bone” MRI was obtained of the phantom in the axial, sagittal and coronal planes. Comparable CT images of the phantom were obtained on a 64 slice Spiral CT scanner (GE Medical Systems, Milwaukee, Ill., USA) with 0.625 mm slice thickness. Axial images were acquired with no gantry tilt and reconstructed in the sagittal and coronal planes. Using an Advantage Windows Workstation (GE Medical Systems, Milwaukee, Ill., USA) the images were reviewed to determine the image on which the anatomical points were most clearly identifiable, and on which the measurements could be made. The image number for each measurement was recorded to ensure the same slice was used for repeated measurements. Each measurement was made using the cursor function, and again repeated twenty times on two separate occasions by one assessor. To minimise bias, distance annotations were removed immediately after each measurement was recorded.

Mann Whitney U Test and Kruskal-Wallis tests were performed using SPSS Version 18 on a Windows PC with the null hypothesis that there was no significant difference between the groups (p<0.05).

Intra-observer concordance was high, with no statistical significance between the two groups of results. The measurements were therefore combined and analysed collectively for each modality.

Using the direct anatomical measurements as a reference group, no statistical difference was found for three of the twelve distances on MRI, and two of the twelve distances on CT (Table 3). Whilst statistically different, the difference between the measurements for each group was less than 1 mm for all measurements (Table 4). The average discrepancy between MRI and anatomical values using the mean for each group was 0.32 mm (0.76%), and for CT 0.40 mm (0.76%). The mean difference between MRI and CT was 0.38 mm.

TABLE 3
Mann-Whitney U Test results highlighting those distances
where there was no significant difference in result

| | Anatomical | Anatomical | CT v |
|---|---|---|---|
| | v MRI | v CT | MRI |
| Right Orbit | <0.05 | <0.05 | <0.05 |
| Left Orbit | 0.11 | 0.48 | 0.22 |
| Mandible Height | <0.05 | <0.05 | 0.42 |
| Coronal Skull Base | <0.05 | <0.05 | 0.11 |
| Coronal Skull Cap | <0.05 | <0.05 | <0.05 |
| Infraorbital | <0.05 | <0.05 | <0.05 |
| Foramina | | | |
| Piriform aperture | <0.05 | 0.27 | <0.05 |
| height | | | |
| Piriform aperture | <0.05 | <0.05 | <0.05 |
| width | | | |
| Mental Foramina | <0.05 | <0.05 | <0.05 |
| Intercondylar | <0.05 | <0.05 | <0.05 |
| Distance | | | |
| Maxilla with teeth | 0.50 | 0.48 | 0.22 |
| Maxilla without | 0.28 | <0.05 | <0.05 |
| teeth | | | |

TABLE 4
Mean and Standard Error results for distances

| | | | | Percentange |
|---|---|---|---|---|
| | | | | difference |
| | | Mean | | compared to |
| | | (mm) | SE | anatomical (%) |
| Right Orbit | Anatomical | 32.74 | 0.038 | |
| | MRI | 32.97 | 0.044 | 0.70 |
| | CT | 32.49 | 0.058 | 0.76 |
| Left Orbit | Anatomical | 32.89 | 0.020 | |
| | MRI | 32.87 | 0.031 | 0.06 |
| | CT | 32.80 | 0.051 | 0.27 |
| Mandible Height | Anatomical | 27.69 | 0.040 | |
| | MRI | 27.99 | 0.053 | 1.08 |
| | CT | 27.93 | 0.054 | 0.87 |
| Coronal Skull Base | Anatomical | 139.48 | 0.040 | |
| | MRI | 139.95 | 0.034 | 0.33 |
| | CT | 140.04 | 0.055 | 0.40 |
| Coronal Skull Cap | Anatomical | 140.62 | 0.045 | |
| | MRI | 140.28 | 0.042 | 0.24 |
| | CT | 140.08 | 0.041 | 0.38 |
| Infraorbital Foramina | Anatomical | 55.81 | 0.008 | |
| | MRI | 56.19 | 0.042 | 0.68 |
| | CT | 56.83 | 0.534 | 1.82 |
| Piriform aperture height | Anatomical | 32.35 | 0.024 | |
| | MRI | 32.39 | 0.038 | 0.12 |
| | CT | 32.96 | 0.042 | 1.89 |
| Piriform aperture width | Anatomical | 21.54 | 0.041 | |
| | MRI | 22.25 | 0.038 | 3.30 |
| | CT | 21.75 | 0.039 | 0.97 |
| Mental Foramina | Anatomical | 44.47 | 0.042 | |
| | MRI | 43.61 | 0.032 | 1.93 |
| | CT | 44.78 | 0.050 | 0.70 |
| Intercondylar Distance | Anatomical | 110.13 | 0.073 | |
| | MRI | 110.40 | 0.086 | 0.25 |
| | CT | 110.93 | 0.037 | 0.73 |
| Maxilla with teeth | Anatomical | 58.11 | 0.061 | |
| | MRI | 58.07 | 0.050 | 0.07 |
| | CT | 58.24 | 0.073 | 0.22 |
| Maxilla without teeth | Anatomical | 40.54 | 0.025 | |
| | MRI | 40.70 | 0.051 | 0.39 |
| | CT | 40.57 | 0.033 | 0.07 |

The “Black Bone” imaging sequence of the present invention therefore provides improved soft-tissue/bone contrast by utilising a low-flip angle to suppress both fat and water to obtain a uniform soft-tissue background. The ability to clearly identify bone is therefore optimised in areas where bone is enveloped within soft tissue, such as the mandibular region. The sequence has been demonstrated to be biometrically comparable to CT.

### Example Diagnostic Applications

The following cases are examples of the diagnostic benefits of the “Black Bone” sequence, offering a non-ionising alternative to CT:

### Patient 1:

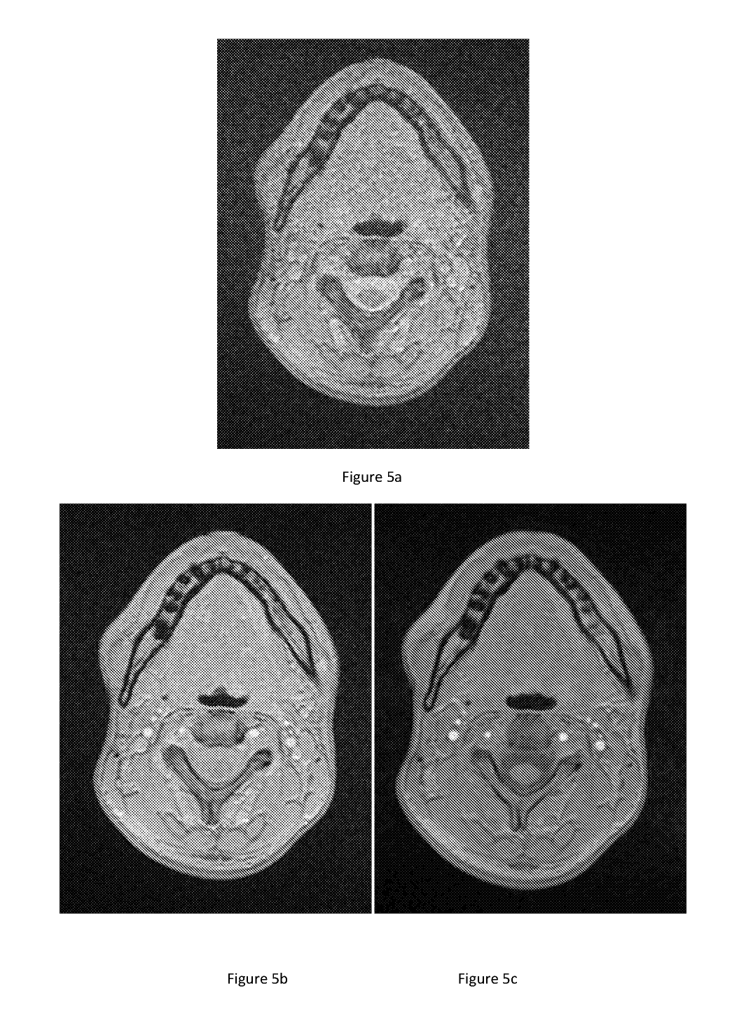

A 31 year old female was referred for CT imaging to investigate increased prominence of the right zygoma. Asymmetry of the facial features had been noted by friends of the patient two months prior to presentation, with no further progression during this period. Of note was an injury to this side of her face in childhood, which required no intervention. Radiography performed by the referring physician was normal. MRI was performed in place of the requested CT for radiation protection. Imaging demonstrated the zygomatic bodies to be within normal limits, with an acute angle between the anterior and lateral walls. The “Black Bone” sequence was useful in demonstrating a normal cortical pattern of the zygomatic bodies, with normal marrow signal from the medulla (FIG. 6). There was no evidence of a mass in the surrounding tissues. However, mucosal thickening in the left maxillary antrum was noted and the medial half of the anterior wall immediately below the orbital rim bulged into the cheek. The patient has been reassured that there is no significant pathology, and remains under review.

### Patient 2

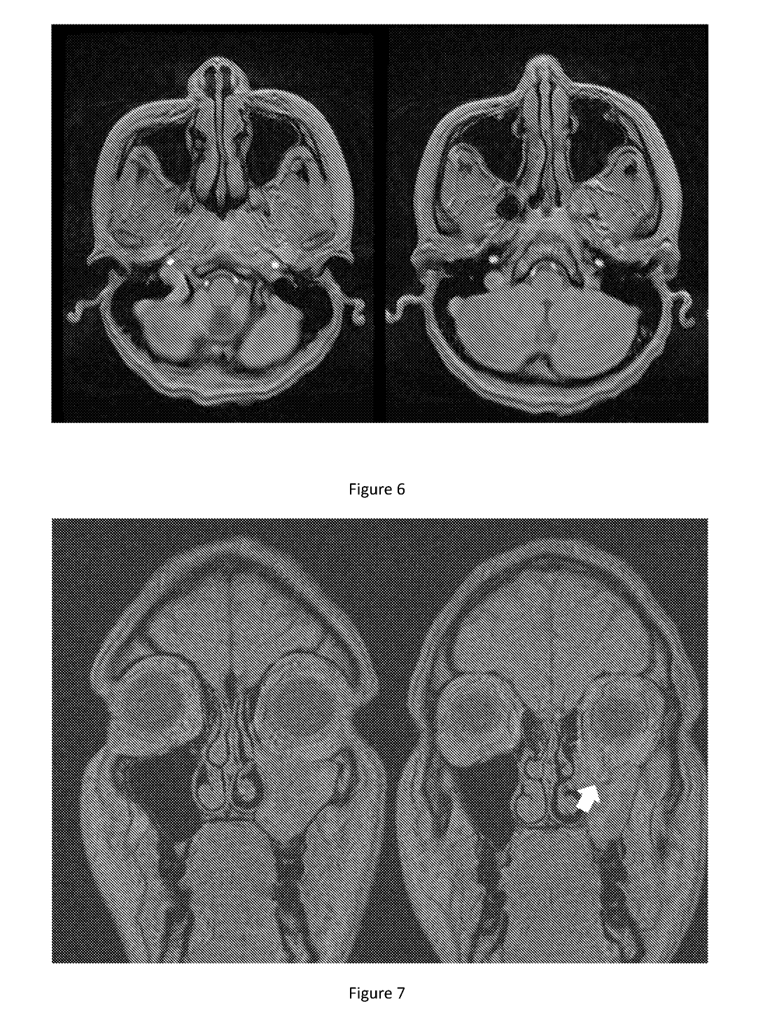

A 38 year old male was referred from the Emergency Department following a rugby injury. He complained of a “popping” sensation in the left globe when blowing his nose. On clinical examination there was a small palpable step in the left infraorbital rim, with no associated paraesthesia or eye symptoms. Facial radiographs performed by the referring team revealed a un-displaced fracture of the left zygoma. With no significant symptoms, the patient opted for non-surgical management, and was discharged. He re-presented 4 weeks later with diplopia on upward gaze. On clinical examination at this time he had mild enophthalmos and hypoglobus of the left orbit. CT imaging of the left orbit was requested but substituted by MRI for radiation protection. MRI demonstrated inferior displacement of almost all of the orbital floor on the left, with a mild degree of enophthalmos. A fracture was noted to extend into the inferior part of the medial wall but with no true medial wall blow-out into the ethmoid sinuses. The underlying antrum was almost completely occupied by extensive mucosal swelling with proteinaceous effusion in the remaining cavity. The “Black Bone” sequence demonstrated that the lateral antral wall was in a normal position and the zygoma appeared to be intact (FIG. 7). He subsequently underwent open reduction and fixation of orbital fracture with autologous bone grafting.

### Patient 3

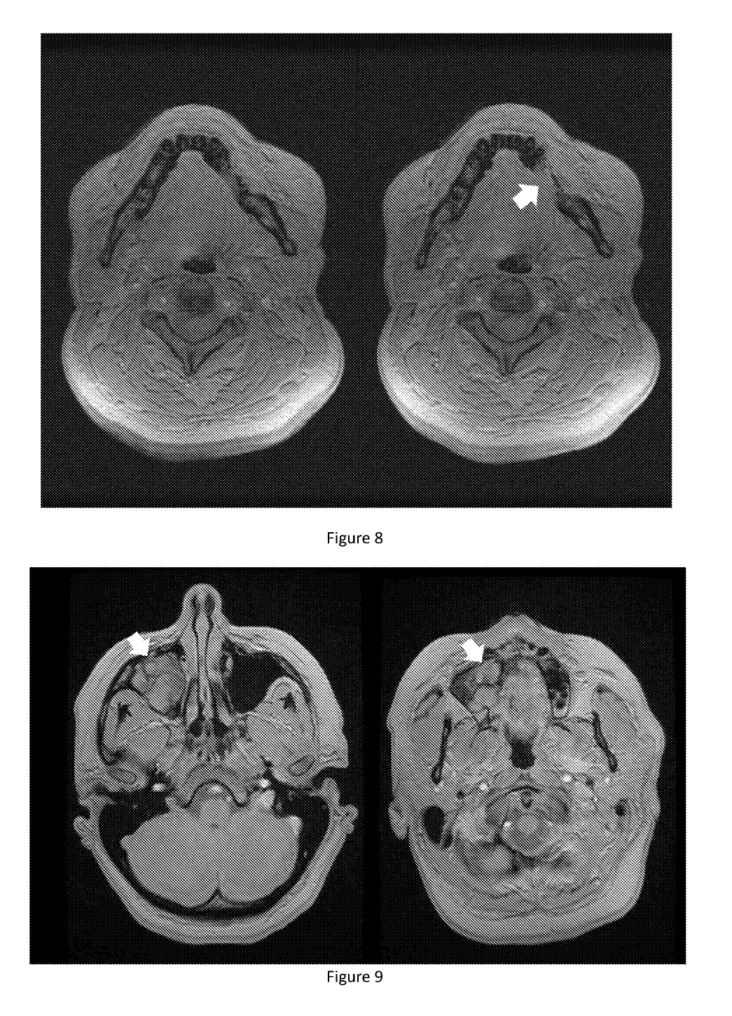

A 62 year old male presented with presumed recurrence of a left mandibular keratocyst, previously excised 16 years earlier. An orthopantogram (OPG) showed radiolucency of the left sigmoid notch and condyle. MRI was performed in place of the requested CT examination. The imaging confirmed a bilobed expansile lesion in the neck of the mandible on the left. This extended into the base of the mandibular head with sparing of a rim of medulla on the superior aspect of the lesion. Overall the lesion measured approximately 2 cm×2 cm×1 cm and extended anteroinferiorly from the mandibular neck into the proximal ramus. The “Black Bone” sequence demonstrated thinning and loss of the cortex on the medial aspect of the lower margin of the lesion and again at its maximum convexity on the medial aspect of the mandible (FIG. 8). There was also a small area of cortical loss on the lateral aspect of the mandibular neck. However there was no detectable extension into the soft tissues.

He underwent further excision of this lesion, with histological examination confirming recurrence of the odontogenic keratocyst.

### Patient 4

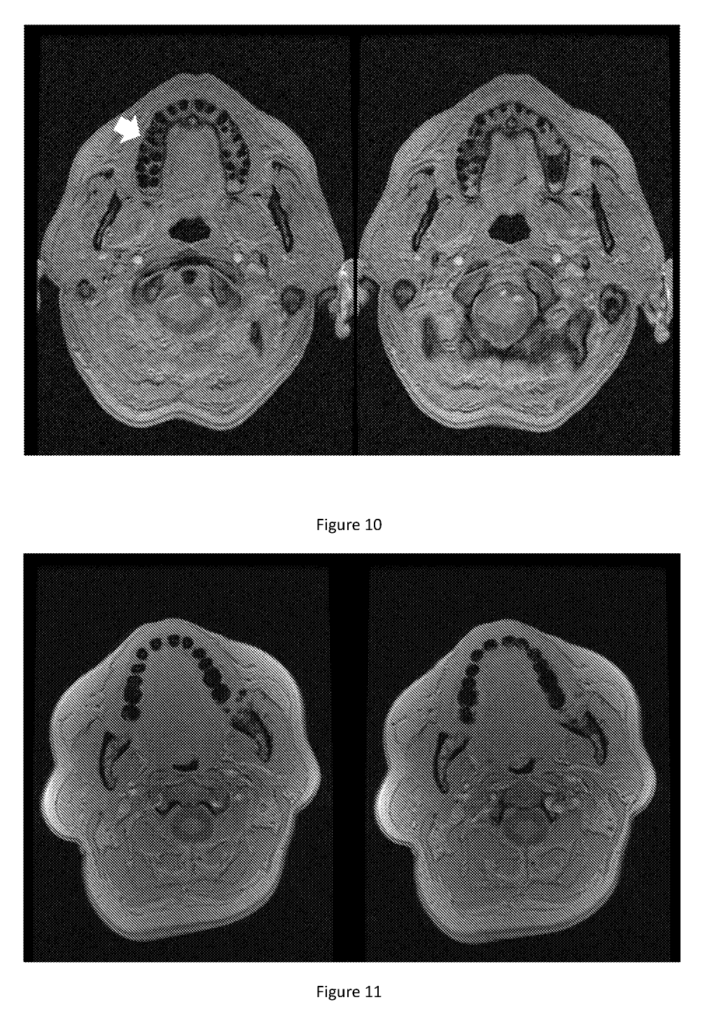

A 46 year old female presented with buccal swelling and loosening of the upper right second premolar and first molar teeth. On OPG there was an area of radiolucency of the maxilla noted, and she underwent exploration of the region, with excision of a mass. Histologically this was confirmed as a giant cell granuloma. Post-operatively she had recurrence of the mass, and was managed with calcitonin therapy, to which she had a good response. Follow up with sequential scanning has been necessary, with preference for MRI for radiation protection. “Black Bone” sequences were of sufficient quality to negate the need for CT (FIG. 9). MR demonstrated minimal expansion of the right antrum, which appeared to be completely occupied by the granuloma. There was expansion of the root of the right superior alveolus without displacement of the cortex or the teeth. She remains under clinical review.

### Patient 5

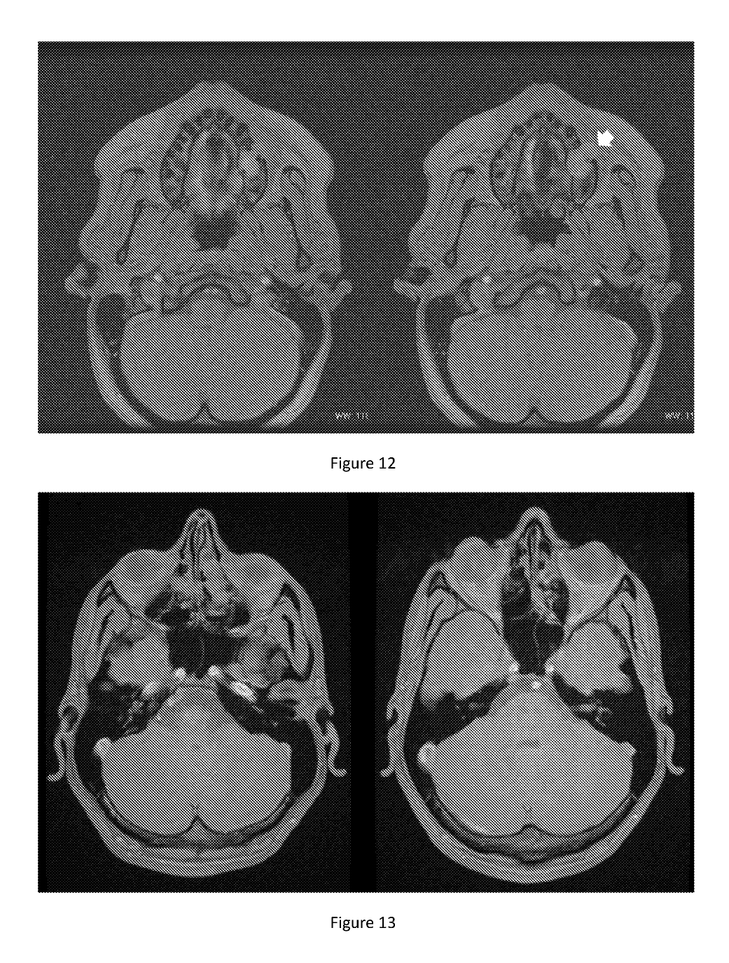

A 58 year old male presented with an intermittently painful lump in the region of the right upper second premolar. This had been present for four months during which time it had gradually increased in size. On examination an 8 mm bony hard lump was noted over the apex of right upper premolar. OPG showed restoration of the second premolar, but no other pathology. A “Black Bone” MRI was specifically requested to investigate this region (FIG. 10). Imaging demonstrated no evidence of bony expansion of the right superior alveolus. There was altered signal around the roots of both right upper fifth and sixth teeth in keeping with periodontal inflammation, and a small effusion in the base of the right maxillary antrum was present. In addition, a well defined signal change was noted around the root of the right lower third tooth, in keeping with apical abscess. Under local anaesthesia the region was explored, and pus found above the disto buccal root of upper first molar tooth, which was curetted out. He was subsequently referred back to his primary care dentist for root canal therapy.

### Patient 6

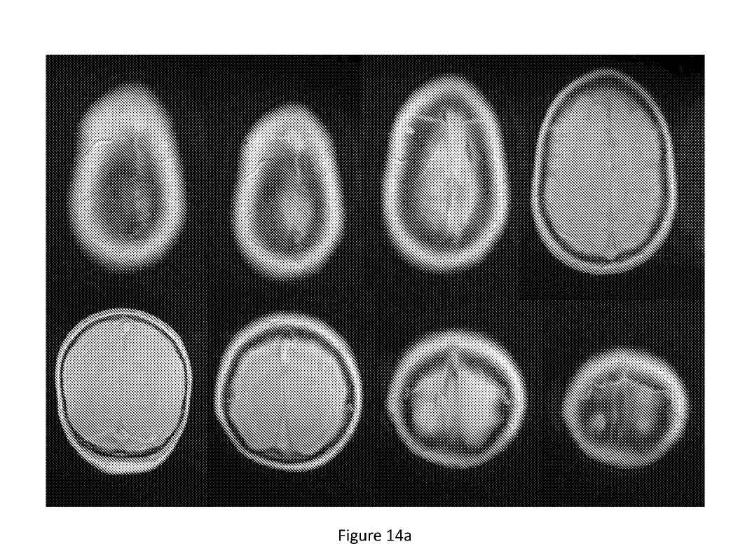

A 56 year old female presented with recurrence of a left retromolar keratocyst. This had previously been enucleated 6 years prior to presentation. She underwent repeat encluceation and histology confirmed an odontogenic keratocyst. Recurrent imaging at 1 year has demonstrated good bony infill with no evidence of cyst recurrence. The “Black bone” sequence demonstrated normal cortical morphology, with evidence of trabecular sclerosis in keeping with healing (FIG. 11). She remains under review.

### Patient 7

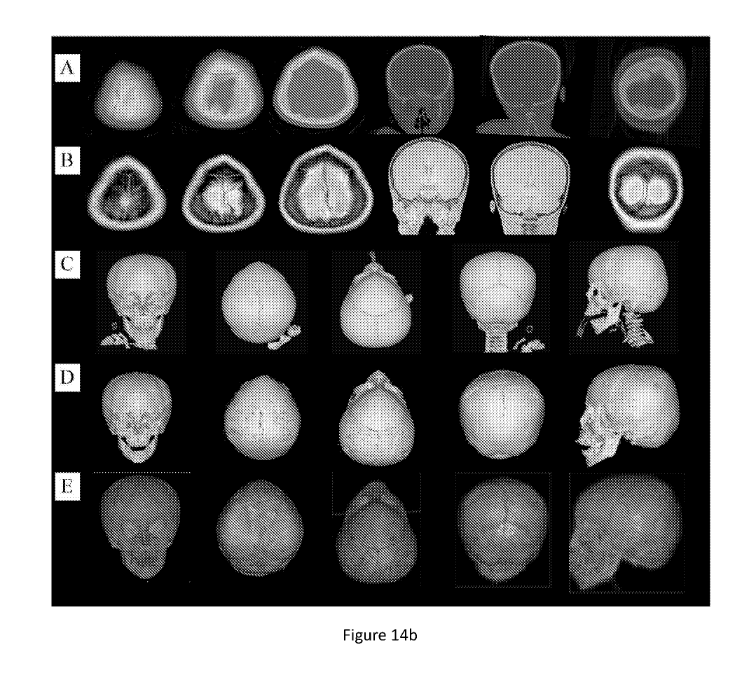

A 26 year old female presented with a three week history of a slowly enlarging non-painful lesion affecting the left upper jaw. Routine OPG demonstrated a radiolucency in the region of the second premolar/first molar. MRI was requested, which demonstrated a high T2 signal lesion arising between the roots of the left upper sixth tooth. “Black bone” imaging demonstrated the lesion extending superiorly into the alveolus and breaching the lateral cortex with a dumbbell extension into the subcutaneous tissues of the cheek (FIG. 12). The findings suggested a slow growing lesion such as a keratocyst or ameloblastoma. A fine needle aspiration of the region confirmed a diagnosis of a benign odontogenic cyst, and the remnants excised following root canal treatment of the adjacent teeth.

### Patient 8

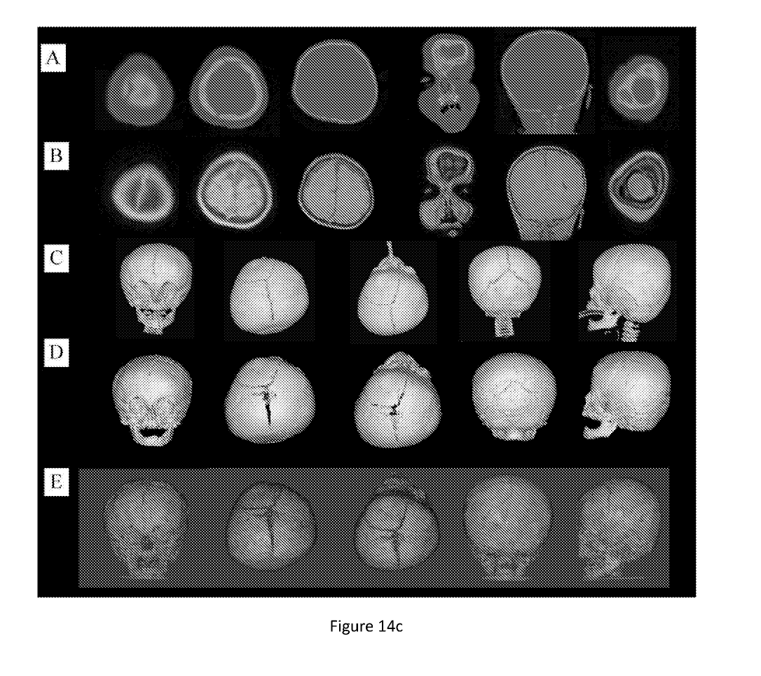

A 23 year old male presented following an alleged assault, having fallen to ground hitting his face. On examination he had mild left infraorbital anaesthesia and slight flattening of the left malar region. Radiographs showed a minimally displaced left zygomatic fracture. He failed to attend follow up, but returned 3 weeks later complaining of worsening diplopia. However, he had a longstanding squint for which he was not currently using his prescription glasses. A HESS chart used to assess diplopia, by demonstrating the position of the non-fixing eye in all positions of gaze when the other eye was fixing, showed no change. Routine MR imaging was requested. Coronal images demonstrated that the left orbital floor was normal. There was no evidence of surrounding fracture on the “Black Bone” images (FIG. 13). He was reassured, and discharged from further follow-up.

All of these presented patients avoided CT examination by utilising the “Black Bone” sequence.

### Craniosynostosis

To investigate the potential of “Black Bone” MRI in the diagnosis of craniosynostosis, the sequence was obtained in children with a clinical diagnosis of craniosynostosis in whom comparable CT imaging was available. The images were independently reviewed to determine if the diagnosis could be accurately made. The normal cranial sutures were visualised as areas of increased signal against the signal void of the bone, and in cases of synostosis, the involved suture was absent on the “Black Bone” images. Independent review demonstrated that the sequence offered considerable potential as an alternative to CT in the diagnosis of craniosynostosis. Examples of images are shown in FIGS. 14a to 14d. FIG. 14a shows axial (top row) and coronal (bottom row) imaging from a child with normal cranial sutures. The cranial sutures are identified as areas of increased signal intensity, easily distinguished from the signal void of the cranial bone.

FIGS. 14b to 14d shows imaging obtained from children with metopic synostosis, right unicoronal synostosis and sagittal synostosis respectively. In each case, row A shows axial and coronal CT images, row B shows axial and coronal MRI images, row C shows 3D CT images, row D shows the 3D “Black Bone” reconstruction using Mimics and row E shows the 3D “Black Bone” reconstruction using Fovia. As will be apparent “Black Bone” MRI can provide useful and accurate identification of cranial sutures and diagnosis of craniosynostosis, and so provides a non-ionising alternative to CT.

The greatest potential of the “Black Bone” sequence of the present invention is in imaging benign conditions of the facial skeleton, since these are frequently necessary in young patient groups and on multiple occasions. The method of the present invention offers a valuable method of radiation dose constraint in such patients.

The method may be used with other imaging techniques, such as PET, providing a rapid acquisition MRI sequence for co-registration with PET.

The method may be used for animal as well as human subjects.

It should be emphasized that the above-described embodiments are merely examples of possible implementations. Many variations and modifications may be made to the above-described embodiments without departing from the principles of the present disclosure. All such modifications and variations are intended to be included herein within the scope of this disclosure and protected by the following claims.

1. A method of obtaining an image of bone using magnetic resonance imaging, the method comprising the step of selecting the scanning parameters for the magnetic resonance imaging device,
wherein the scanning parameters comprise a low flip angle, a short time to repetition and a short time to echo.

2. The method of claim 1, wherein the flip angle is in the range 1° to 10°.

3. The method of claim 2 wherein the flip angle is one of about 1°, 3°, 5° and 7°

4. The method of claim 1, wherein the time to repetition is ≦20 ms.

5. The method of claim 1, wherein the time to echo is <20 ms.

6. The method of claim 1, wherein the time to echo is one of the following: 4.6; 9.2; 13.8; and 18.4 ms.

7. The method of claim 1 wherein the magnetic resonance imaging uses a gradient echo sequence.

8. The method of claim 1, wherein the time to echo is “in phase”.

9. The method of claim 1 comprising performing 3D reconstruction using the image data.
